(19) United States
(12) Patent Application Publication
(10) Pub. No.: US 2021/0161501 A1
(43) Pub. Date: Jun. 3, 2021

(54) RADIOGRAPHY APPARATUS

(71) Applicant: FUJIFILM Corporation, Tokyo (JP)

(72) Inventor: Tomonari SENDAI, Ashigarakami-gun (JP)

(73) Assignee: FUJIFILM Corporation, Tokyo (JP)

(21) Appl. No.: 17/176,240

(22) Filed: Feb. 16, 2021

Related U.S. Application Data
(63) Continuation of application No. PCT/JP2019/032167, filed on Aug. 16, 2019.

(30) Foreign Application Priority Data
Aug. 17, 2018 (JP) 2018-153734

(51) Int. Cl. A61B 6/00; A61B 6/06

(52) U.S. Cl. CPC A61B 6/547; A61B 6/544; A61B 6/5211; A61B 6/06

(57) ABSTRACT

A radiography apparatus includes a radiation source, a radiation detection unit, an imaging unit, a first recognition unit that recognizes the radiation detection unit, a second recognition unit that recognizes a subject using an image captured by the imaging unit, a determination unit that, using recognition results of the first recognition unit and the second recognition unit, specifies a relative positional relationship between the radiation detection unit and the subject and determines whether or not the subject is in a detection effective region of the radiation detection unit, and a controller that performs operation support using a determination result of the determination unit.

### CROSS-REFERENCE TO RELATED APPLICATIONS

This application is a Continuation of PCT International Application No. PCT/JP2019/032167 filed on 16 Aug. 2019, which claims priority under 35 U.S.C. § 119(a) to Japanese Patent Application No. 2018-153576 filed on 17 Aug. 2018. The above application is hereby expressly incorporated by reference, in its entirety, into the present application.

### BACKGROUND OF THE INVENTION

### 1. Field of the Invention

The present invention relates to a radiography apparatus that images a subject using radiation, such as X-rays.

### 2. Description of the Related Art

A radiography apparatus comprises a radiation source that generates radiation, and a radiation detection unit that detects radiation. A subject is disposed between the radiation source and the radiation detection unit. Then, the radiography apparatus acquires an image of the subject (in particular, the inside of the subject) by detecting radiation transmitted through the subject with the radiation detection unit.

In the radiography apparatus, the radiation source and the radiation detection unit may be movable independently. For example, in a mobile X-ray imaging apparatus (so-called treatment cart), a radiation source and a radiation detection unit are movable independently. In a case where the radiation source and the radiation detection unit are movable independently, alignment of the radiation source and the radiation detection unit is required at the time of imaging. In recent years, a radiography apparatus that detects a position of a radiation detection unit and moves a radiation source in conformity with the position of the radiation detection unit is known (JP2015-156896A and JP1999-276463A (JP-H11-276463A)).

### SUMMARY OF THE INVENTION

In a case where the radiation source and the radiation detection unit are movable independently, alignment of at least the radiation source and the radiation detection unit is required. Meanwhile, merely aligning relative positions of the radiation source and the radiation detection unit may cause failure in imaging. This is because a subject can be discretionarily disposed between the radiation source and the radiation detection unit. Specifically, in a case where the subject is not in a detection effective region of the radiation detection unit, even though the radiation source and the radiation detection unit are aligned, there is a problem in that the whole of the subject is not captured in an acquired radiographic image. For this reason, it is desirable that a radiography apparatus, in which a radiation source and a radiation detection unit are movable independently, supports imaging in consideration of disposition of a subject.

An object of the invention is to provide a radiography apparatus that supports reliable imaging even though a radiation source and a radiation detection unit are movable independently.

A radiography apparatus according to an aspect of the invention comprises a radiation source that generates radiation, a radiation detection unit that is movable independently with respect to the radiation source and obtains an image of a subject by detecting the radiation transmitted through the subject, an imaging unit that images at least the subject using light having a wavelength longer than the radiation, a first recognition unit that recognizes the radiation detection unit, a second recognition unit that recognizes the subject using an image captured by the imaging unit, a determination unit that, using recognition results of the first recognition unit and the second recognition unit, specifies a relative positional relationship between the radiation detection unit and the subject and determines whether or not the subject is in a detection effective region of the radiation detection unit, and a controller that performs operation support using a determination result of the determination unit.

It is preferable that the controller notifies that the subject is in the detection effective region or that the subject is not in the detection effective region.

It is preferable that, in a case where the subject is not in the detection effective region, the controller notifies that the subject is not in the detection effective region after the second recognition unit recognizes the subject.

In a case where the subject is not in the detection effective region and the subject is not recognizable by the second recognition unit, the controller notifies that the subject is not recognizable.

It is preferable that the controller validates or invalidates exposure of the radiation using the determination result.

It is preferable that the controller validates the exposure of the radiation in a case where the subject is in the detection effective region.

It is preferable that the controller invalidates the exposure of the radiation in a case where the subject is not in the detection effective region.

It is preferable that the controller performs operation support of position adjustment for putting the subject in the detection effective region in a case where the subject is not in the detection effective region.

It is preferable that the controller notifies of a direction for moving the radiation detection unit to put the subject in the detection effective region.

It is preferable that the controller notifies of a distance for moving the radiation detection unit to put the subject in the detection effective region.

It is preferable that the controller notifies of an orientation, a direction, or an angle for rotating or inclining the radiation detection unit to put the subject in the detection effective region.

It is preferable that the radiography apparatus further comprises a collimator that defines an irradiation field of the radiation, and the controller performs control using the determination result that the collimator adjusts the irradiation field.

It is preferable that the radiography apparatus further comprises a collimator that defines an irradiation field of the radiation, and the controller puts the subject in the detection effective region by performing control using the determination result such that the collimator changes the irradiation field.

It is preferable that the radiography apparatus further comprises a collimator that defines an irradiation field of the radiation, and the controller performs control such that the collimator changes the irradiation field in conformity with the subject in a case where the subject is in the detection effective region.

It is preferable that the first recognition unit specifies a position and an orientation of the radiation detection unit.

It is preferable that the first recognition unit recognizes the radiation detection unit using the image acquired by the imaging unit.

It is preferable that the radiation detection unit has a marker indicating a position, and the first recognition unit recognizes a position and an orientation of the radiation detection unit using the marker in the image acquired by the imaging unit.

It is preferable that the radiation detection unit comprises a position sensor that measures a position of the radiation detection unit, and the first recognition unit recognizes the radiation detection unit using information obtained with the position sensor.

It is preferable that the second recognition unit recognizes a part of the subject.

It is preferable that the second recognition unit recognizes the part of the subject by matching of an image of the subject captured by the imaging unit and a template.

It is preferable that the second recognition unit is artificial intelligence.

It is preferable that the controller automatically moves the radiation source to a position confronting the radiation detection unit in a case where the first recognition unit recognizes the radiation detection unit.

It is preferable that the controller automatically moves the radiation source in a case where the radiation detection unit is horizontal and stationary.

It is preferable that, in a case of automatically moving the radiation source, the controller notifies of an effect that the radiation source is to be moved.

It is preferable that the controller maintains a distance between the radiation source and the radiation detection unit at a specific distance.

It is preferable that the controller moves the imaging unit in a case where the second recognition unit does not recognize the subject.

It is preferable that the radiography apparatus further comprises a body thickness measurement unit that measures a body thickness of the subject, and the controller performs operation support using the determination result and the body thickness of the subject.

It is preferable that the controller sets an imaging condition using the body thickness of the subject in a case where the subject is in the detection effective region.

It is preferable that the controller sets a tube voltage of the radiation source using the body thickness of the subject.

It is preferable that the controller inserts or extracts a filter shielding a part of the radiation between the radiation source and the subject using the body thickness of the subject.

It is preferable that, in a case of manually moving the radiation source, the controller notifies that the radiation source is at a position confronting the radiation detection unit.

It is preferable that, in a case of manually moving the radiation source, the controller notifies of a direction for moving the radiation source.

It is preferable that, in a case where the radiation detection unit is attached to a main body including the controller, the controller restricts movement of the radiation source.

It is preferable that, in a case where the radiography apparatus is a mobile type and a main body including the controller is moving, the controller restricts movement of the radiation source.

According to the aspect of the invention, it is possible to provide a radiography apparatus that supports reliable imaging even though a radiation source and a radiation detection unit are movable independently.

### BRIEF DESCRIPTION OF THE DRAWINGS

FIG. 1 is a perspective view of a radiography apparatus.

FIG. 2 is a perspective view of the radiography apparatus.

FIG. 3 is a perspective view of the radiography apparatus in which an arm portion is folded.

FIG. 4 is a plan view showing the configuration of a distal end portion.

FIG. 5 is an explanatory view of markers that are provided on a surface of a radiation detection unit.

FIG. 6 is a block diagram of the radiography apparatus.

FIG. 7 is an explanatory view of template matching that is performed by a second recognition unit.

FIG. 8 is a block diagram of a controller.

FIG. 9 is a flowchart showing an operation aspect of the radiography apparatus.

FIG. 10 is an explanatory view showing disposition at the time of imaging.

FIG. 11 is an explanatory view showing disposition of the radiation detection unit and a subject on a bed.

FIG. 12 is a display example of a touch panel.

FIG. 13 is a block diagram of a radiography apparatus of a second embodiment.

FIG. 14 is a flowchart showing an operation aspect of the radiography apparatus of the second embodiment.

FIG. 15 is a display example showing an aspect of supporting re-disposition of a radiation source.

FIG. 16 is a display example showing an aspect of supporting re-disposition of the radiation source.

FIG. 17 is a perspective view of a radiography apparatus having a telescopic arm.

### DESCRIPTION OF THE PREFERRED EMBODIMENTS

### First Embodiment

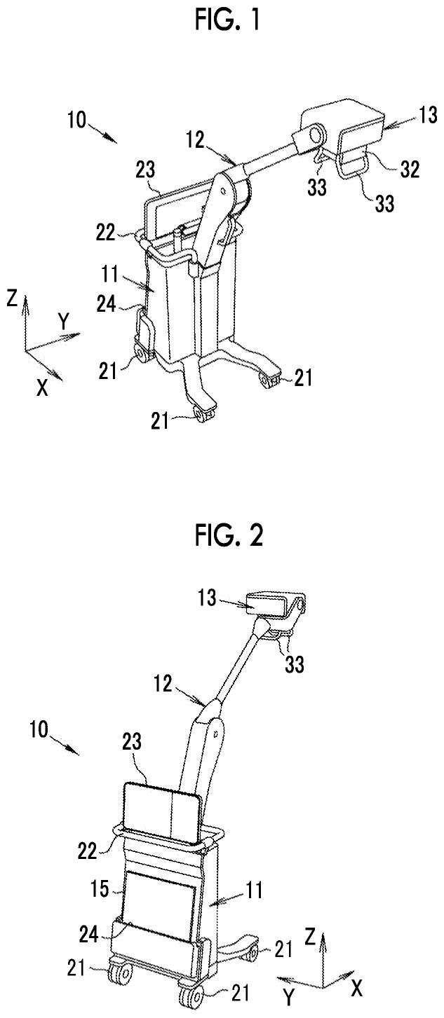

As shown in FIGS. 1 and 2, a radiography apparatus 10 comprises a main body 11, an arm portion 12, a distal end portion 13, and a radiation detection unit 15.

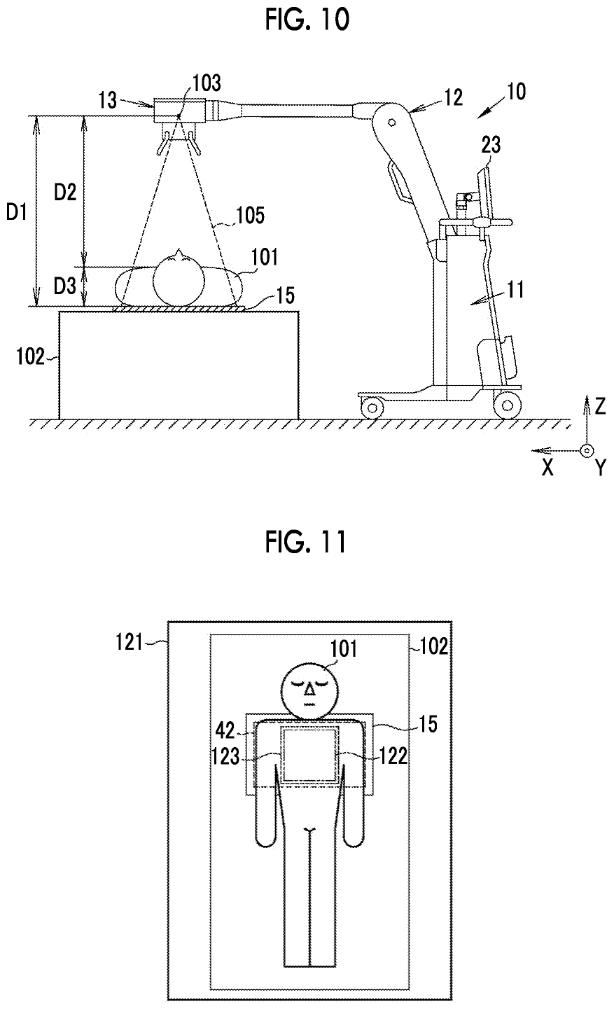

The main body 11 incorporates a control substrate or the like that controls each portion of the radiography apparatus 10, such as the arm portion 12 and the distal end portion 13. The radiography apparatus 10 is a mobile type (so-called treatment cart), and casters 21 are attached to the main body 11. For this reason, it is possible to not only perform radiography not only in a specific laboratory but also move the radiography apparatus 10 to a patient's room where a patient 101 (see FIG. 10) as a subject is present and perform radiography in the patient's room. The casters 21 are rotatable manually or automatically according to settings or the like (selection of an operation mode, and the like). That is, a laboratory technician, a physician, other medical staff, or the like (hereinafter, referred to as a laboratory technician or the like) can manually move the radiography apparatus 10 by pushing and pulling the radiography apparatus 10. Furthermore, the radiography apparatus 10 can automatically move or adjust a position of the apparatus (main body 11). The main body 11 is provided with a grip portion 22 that is gripped by the laboratory technician or the like in manually moving the radiography apparatus 10.

The main body 11 comprises a touch panel 23. The touch panel 23 is a display unit of the radiography apparatus 10 and is an operating unit. The touch panel 23 displays an image of a subject obtained by radiography, items pertaining to operations and settings, and the like. In regard to the items pertaining to the operations and the settings, the laboratory technician or the like can perform an input of settings, an input of an operation instruction, and the like to the radiography apparatus 10 by touching the touch panel 23 with a finger or the like.

In addition, the main body 11 has an insertion type holder 24 in which the radiation detection unit 15 is put. Inside the holder 24, a sensing unit (not shown) that senses insertion or non-insertion of the radiation detection unit 15 is provided. For this reason, the radiography apparatus 10 can sense insertion and extraction of the radiation detection unit 15. The sensing unit is, for example, a switch that is turned on in a case where the radiation detection unit 15 is held in the holder 24 and is turned off in a case where the radiation detection unit 15 is extracted from the holder 24.

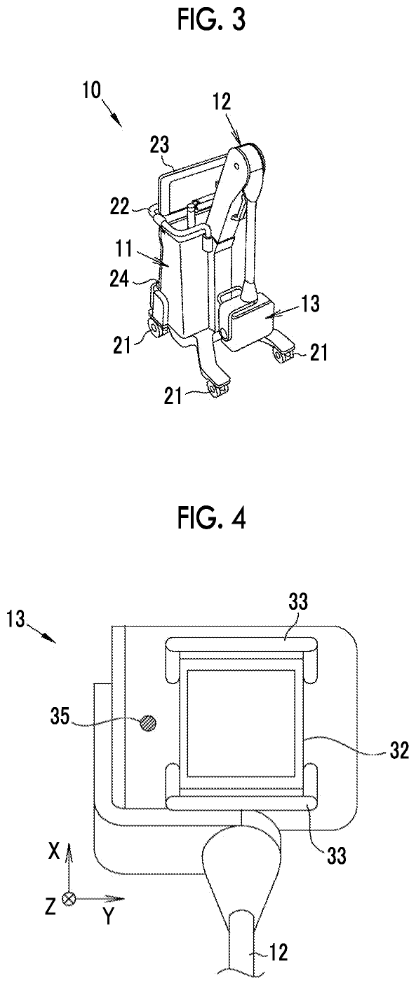

The arm portion 12 holds the distal end portion 13 with respect to the main body 11. The arm portion 12 is foldable. In a case where the radiography apparatus 10 is moved to a patient's room or the like, the arm portion 12 is folded as shown in FIG. 3, and in a case where imaging is performed, the arm portion 12 is expanded (see FIG. 1 or the like). The radiography apparatus 10 can dispose the position of the distal end portion 13 at a position (hereinafter, referred to as an imaging position) for use in imaging by adjusting an expansion angle of the arm portion 12. The arm portion 12 has a lock mechanism (not shown) and can maintain any expansion angle. For this reason, in a case where imaging is performed, the arm portion 12 can maintain the position of the distal end portion 13 at the imaging position. Folding and expanding of the arm portion 12 can be performed manually or automatically according to settings.

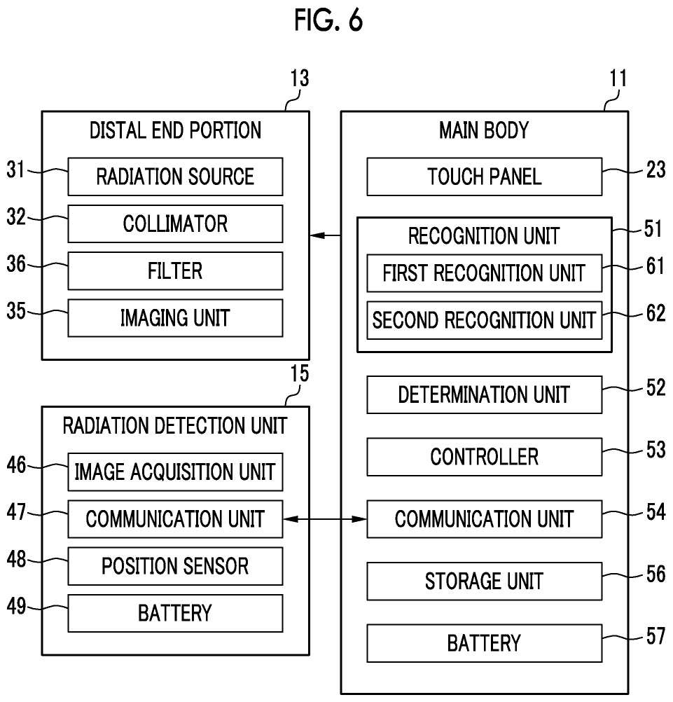

The distal end portion 13 is attached to a distal end of the arm portion 12 to be rotationally movable with respect to the arm portion 12, and incorporates a radiation source 31 (see FIG. 6) that generates radiation. The distal end portion 13 has a collimator 32 that defines an irradiation field 105 (see FIG. 10) of radiation, and one or a plurality of handles 33.

The radiation source 31 is an X-ray tube in the embodiment. For this reason, the radiography apparatus 10 is an X-ray imaging apparatus that images a subject using X-rays. Energy, a dose, or the like of radiation generated by the radiation source 31 is one of imaging conditions in radiography and can be set manually or automatically. In the embodiment, the energy of the exposure X-rays can be changed by adjusting a tube voltage of the X-ray tube. The dose of the exposure X-rays can be changed by adjusting a tube current of the X-ray tube. As the radiation source 31, a radiation source that generates radiation (gamma-rays or the like) other than X-rays can be used.

The collimator 32 incorporates one or a plurality of shielding plates (not shown) that shield radiation generated by the radiation source 31, and adjusts the size and shape of the irradiation field of exposure radiation by adjusting at least one of a position or an orientations (angle) of each shielding plate. For example, the collimator 32 has four shielding plates. The collimator 32 forms the shape of exposure radiation in a rectangular shape and adjusts the size of the rectangular shape with the four shielding plates. The adjustment of the irradiation field 105 using the collimator 32 can be performed manually or automatically according to settings. The irradiation field 105 of radiation is one of imaging conditions in radiography.

The handle 33 is gripped, for example, in a case where the laboratory technician or the like manually moves the distal end portion 13, or the like. The handle 33 also functions as a guard that maintains a minimum distance distal end portion 13 and the subject in a case where the distal end portion 13 is disposed close to the subject.

In addition, as shown in FIG. 4, the distal end portion 13 has an imaging unit 35. The imaging unit 35 images at least the subject using light having a wavelength longer than radiation generated by the radiation source 31. Light having a wavelength longer than radiation generated by the radiation source 31 is, for example, ultraviolet rays, visible light, infrared rays, or the like. Accordingly, the imaging unit 35 is a camera that images the subject or the like using electromagnetic waves or the like other than radiation. More specifically, the imaging unit 35 is, for example, a digital camera, a video camera, or the like.

An imaging range of the imaging unit 35 includes at least an exposure range of radiation generated by the radiation source 31. This is because an image (hereinafter, referred to as a camera image 121; see FIG. 10) captured by the imaging unit 35 is used in recognition processing of recognizing the subject or the like, and as a result, is used to determining whether or not the subject is in a detection effective region of the radiation detection unit.

The subject that is at least used by the imaging unit 35 as an imaging target is a subject, such as a patient, to be imaged by the radiography apparatus 10 using radiation. The imaging unit 35 may use the radiation detection unit 15 as an imaging target. This is because the radiography apparatus 10 may use an image obtained with the imaging unit 35 in recognition processing of recognizing the radiation detection unit 15.

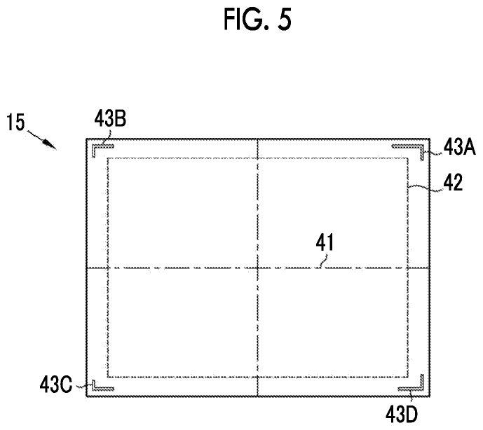

The radiation detection unit 15 is movable independently with respect to the radiation source 31 and obtains an image (hereinafter, referred to as a radiographic image) of the subject by detecting radiation transmitted through the subject. Obtaining the radiographic image with the radiation detection unit 15 is referred to as radiography. In the embodiment, a so-called flat panel detector (FPD) is applied. As shown in FIG. 5, in the radiation detection unit 15, markers indicating positions, such as at least one of a marker 41 indicating the position of the center of the detection effective region, a marker 42 indicating a position and a range of the detection effective region, or markers 43A to 43D indicating positions of corner portions of the radiation detection unit 15, are appropriately provided. This is to allow at least one of the laboratory technician, the radiography apparatus 10, or the like to easily recognize a position and an orientation of the radiation detection unit 15, the detection effective region, and the like. The detection effective region is a region where there are pixels contributing to a radiographic image and detection of radiation is actually effective. The markers 43A to 43D can be identified in distinction from one another using a shape, a color, or the like. In the embodiment, the markers 43A to 43D are an L shape, and the markers 43A to 43D are different in length of a side of the L shape for identification.

As shown in FIG. 6, the distal end portion 13 comprises a filter 36 in addition to the radiation source 31, the collimator 32, and the imaging unit 35. The filter 36 is a member that shields a part of radiation generated by the radiation source 31, and is insertable and extractable into and from an exposure path of radiation generated by the radiation source 31. The filter 36 is, for example, a thin plate made of copper or the like, and in the embodiment, primarily shields a low energy component out of radiation generated by the radiation source 31. For this reason, in a case where the filter 36 is inserted into the exposure path of radiation, compared to a case where the filter 36 is not used, the low energy component of radiation decreases, and radiation containing a relatively large amount of high energy component reaches the subject and the radiation detection unit 15.

The radiation detection unit 15 comprises a battery 49 or the like that supplies electric power to an image acquisition unit 46, a communication unit 47, a position sensor 48, and each unit of the radiation detection unit 15.

The image acquisition unit 46 receives radiation to acquire a radiographic image. In a case where the radiation detection unit 15 is a so-called indirect conversion type, the image acquisition unit 46 includes a scintillator that converts radiation into an optical signal once, and then, converts the optical signal into an electrical signal, such as gadolinium oxide sulfur (GOS) or cesium iodide (CsI), thin film transistor (TFTs), and the like. In a case where the radiation detection unit 15 is a so-called direct conversion type, the image acquisition unit 46 includes amorphous selenium or the like that directly converts radiation into an electrical signal, TFTs, and the like.

The communication unit 47 performs communication with the main body 11 in a wired or wireless manner and transmits and receives various control signals and the like. The communication unit 47 transmits the radiographic image acquired by the image acquisition unit 46 to the main body 11.

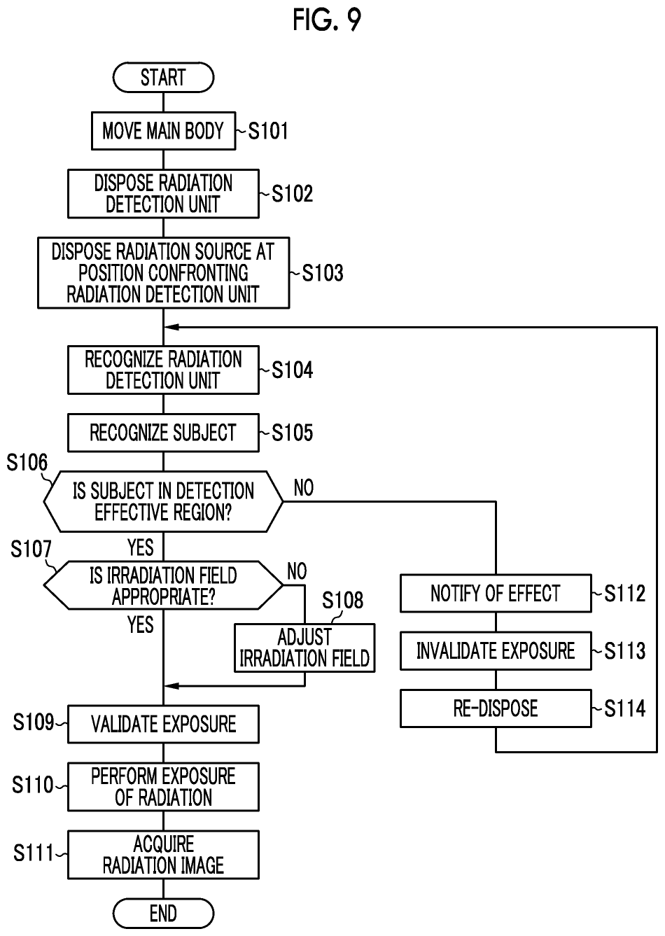

The position sensor 48 is a sensor that detects the position and the orientation (including a direction of change or a change amount of the position or the orientation) of the radiation detection unit 15. The position sensor 48 is, for example, a gyro sensor that detects an angular velocity or an angular acceleration, a speed sensor that detects a speed, an acceleration sensor that detects an acceleration, or a combination thereof. The position of the radiation detection unit 15 is a position with respect to the main body 11, and is, for example, a position with respect to, in particular, a generation point 103 (so-called focus; see FIG. 9) of radiation by the radiation source 31. The orientation of the radiation detection unit 15 is a spatial rotation angle and an inclination angle of the radiation detection unit 15. In the embodiment, the main body 11 traces the radiation detection unit 15 by recognizing the position and the orientation of the radiation detection unit 15 using information (for example, a signal output directly from the position sensor 48) obtained with the position sensor 48. Note that the position sensor 48 can include a measurement unit that measures the position and the orientation of the radiation detection unit 15 using signals output directly from various sensors. In this case, the position sensor 48 can directly output information regarding the position and the orientation of the radiation detection unit 15.

The main body 11 comprises a recognition unit 51, a determination unit 52, a controller 53, a communication unit 54 that performs communication with the communication unit 47 of the radiation detection unit 15, a storage unit 56 stores the radiographic image and the like acquired from the radiation detection unit 15, and a battery 57 that supplies electric power to each unit of the main body 11 and the like.

The recognition unit 51 recognizes a part or the whole of at least one of the subject or the radiography apparatus 10. The recognition that is performed by the recognition unit 51 refers to specification of a spatial position, an orientation, a size, a shape, and the like. In the embodiment, the recognition unit 51 comprises at least a first recognition unit 61 and a second recognition unit 62.

The first recognition unit 61 recognizes the radiation detection unit 15. The recognition of the radiation detection unit 15 refers to specification of (calculating or the like) a position and an orientation of the radiation detection unit 15 with respect to the main body 11. In the embodiment, the first recognition unit 61 specifies a position and an orientation of the radiation detection unit 15 with the generation point 103 of radiation as a reference. The first recognition unit 61 recognizes the radiation detection unit 15 using at least one of the camera image 121 captured by the imaging unit 35 or an output signal or the like of the position sensor 48. Since a position and an imaging range of the imaging unit 35 in the main body 11 are known, the first recognition unit 61 can recognize the radiation detection unit 15 using a position of at least one of an edge (side), an apex (angle), the marker 41, the marker 42, the markers 43A to 43D, or the like of the radiation detection unit 15 in the camera image 121. The first recognition unit 61 can recognize the radiation detection unit 15 by specifying the position and the orientation of the radiation detection unit 15 using the output signal or the like of the position sensor 48. In the embodiment, in principle, the first recognition unit 61 recognizes the radiation detection unit 15 by combining the camera image 121 and the output signal or the like of the position sensor 43. Then, in a case where the radiation detection unit 15 is not captured in the camera image 121 to a degree enough for recognition, or the like, the first recognition unit 61 recognizes the radiation detection unit 15 using the output signal or the like of the position sensor 43.

The recognition of the radiation detection unit 15 in the first recognition unit 61 includes recognition (specification of at least one of a position or an orientation) of the detection effective region of the radiation detection unit 15. This is because the detection effective region is determined in advance for each radiation detection unit 15 and is known, and thus, the recognition of the radiation detection unit 15 is substantially synonymous with the recognition of the detection effective region of the radiation detection unit 15.

The second recognition unit 62 recognizes the subject. The recognition of the subject refers to specification of at least one of a position, an orientation, a shape, a size, or the like of the subject. The recognition of the subject includes recognition of a part of the subject in addition to recognition of the whole subject. That is, the second recognition unit 62 can recognize a part of the subject. Specifically, the second recognition unit 62 can specify at least one of a position, an orientation, a shape, a size, or the like of a part (for example, a chest (portion) with respect to the patient (whole); hereinafter, referred to as an imaging part) for capturing a radiographic image in the subject. In the embodiment, the second recognition unit 62 specifies a position or the like of the subject with the generation point 103 of radiation as a reference. This is to uniformize the reference with the recognition of the radiation detection unit 15 in the first recognition unit 61.

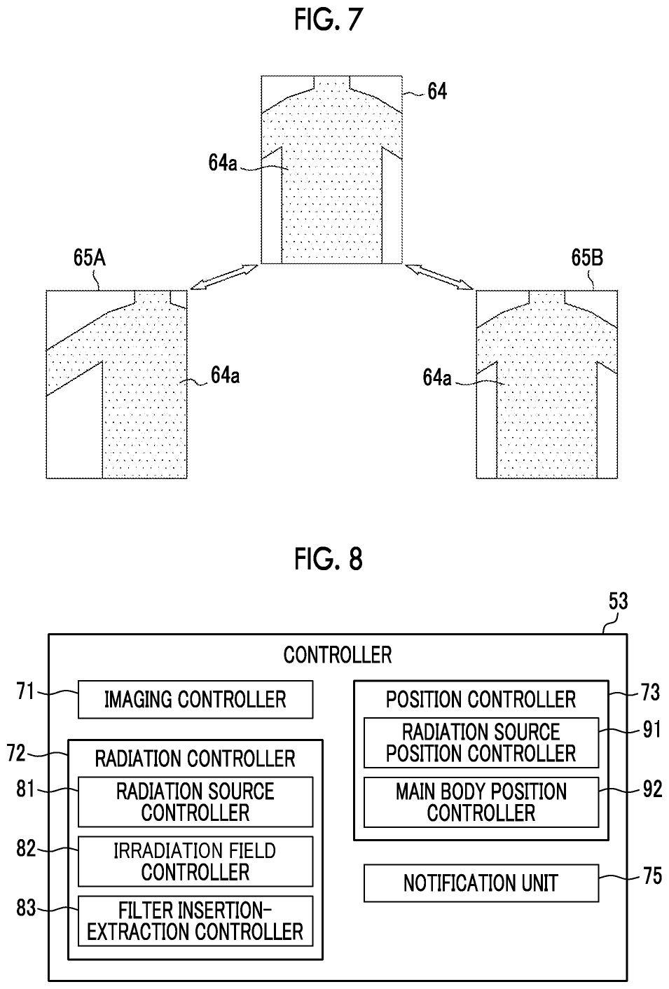

The second recognition unit 62 recognizes the subject using the camera image 121 captured by the imaging unit 35. More specifically, the second recognition unit 62 recognizes the subject (in the embodiment, the imaging part) by matching (so-called template matching) of the camera image 121 of the subject captured by the imaging unit 35 and a template. The template is, for example, the camera image 121 in a case where a radiographic image without excess and deficiency is obtained on a specific imaging part. The second recognition unit 62 has a plurality of kinds of templates according to characteristics, such as at least one of age, sex, or physique of the subject, and recognizes the subject by matching using an appropriate template depending on to the characteristics of the subject or by round robin matching with the templates held therein. Matching refers to obtaining correlation with a template. The second recognition unit 62 specifies the position of the subject or the imaging part of the subject in the camera image 121 using the magnitude of the correlation with the template. As shown in FIG. 7, for example, the second recognition unit 62 compares a template 64, in which a chest 64a of the patient is captured, with a part of the camera image 121 (the whole of the camera image 121 depending on the imaging range) to obtain correlation. Like a comparison range 65A as a part of the camera image 121, in a case where the subject significantly deviates from the subject captured in the template 64, or the like, the correlation is small. On the other hand, like a comparison range 65B, in a case where the position or the like of the subject substantially coincides with the template 64, the correlation is large. For this reason, the second recognition unit 62 can specify that the subject (in particular, the chest as the imaging part) is present in the comparison range 65B having large correlation or a portion in the vicinity.

The second recognition unit 62 changes a template to be used according to an imaging menu. Specifically, in a case where there is a setting of an imaging menu capable of specifying the imaging part, the second recognition unit 62 changes a template to be used according to the setting. The imaging menu is a setting pertaining to at least one of the subject or an imaging condition. For example, the second recognition unit 62 can appropriately use a template of a chest, a head, an abdomen, a limb, or the like. In a case where there is a setting of an imaging menu capable of specifying the characteristic of the subject, such as sex or age, the second recognition unit 62 changes a template to be used according to the setting. For example, the second recognition unit 62 appropriately uses a template for male and a template for female. For example, the second recognition unit 62 appropriately uses a template for aged person, a template for adult (for ordinary adult excluding aged person and child), or a template for child. In this way, the second recognition unit 62 can particularly accurately recognize the position or the like of the subject by appropriately using a plurality of kinds of templates according to the setting of the imaging menu.

In a case where an imaging menu is not used in template switching, such as a case where there is no imaging menu or a case where recognition processing of the second recognition unit 62 is not associated with the imaging menu, the second recognition unit 62 takes correlation using one or a plurality of representative templates among a plurality of templates held therein and decides a template to be used based on the template having the largest correlation. For example, in a case where the irradiation field 105 and the vicinity are captured in the camera image 121, an imaging part can be specified by taking correlation with templates for adult of a chest, a head, an abdomen, and a limb. The same applies to the sex, age, or the like of the subject. In this case, the correlation with the representative templates is taken, whereby the second recognition unit 62 can finally properly select a template to be used.

The second recognition unit 62 can be constituted of artificial intelligence (AI) that has learned using an algorithm of machine learning or deep learning, such as a neural network (NN), a convolutional neural network (CNN), adaboost, or Random Forest, instead of the above-described template matching. In this case, training data (so-called OK image) of correct answer is, for example, the camera image 121 in a case where a radiographic image without excess and deficiency is obtained on a specific imaging part. Training data (so-called NG image) of incorrect answer is, for example, the camera image 121 in a case where a radiographic image with excess and deficiency is obtained.

Using recognition results of the first recognition unit 61 and the second recognition unit 62, the determination unit 52 specifies a relative positional relationship between the radiation detection unit 15 and the subject and determines whether or not the subject is in the detection effective region of the radiation detection unit 15. A determination reference regarding whether or not the subject is in the detection effective region of the radiation detection unit 15 is the generation point 103 of radiation and the irradiation field 105 of radiation. That is, “the subject is in the detection effective region of the radiation detection unit 15” refers to a positional relationship in which the imaging part of the subject is within the irradiation field 105 of radiation and radiation transmitted through the imaging part of the subject reaches the detection effective region. “The subject is not in the detection effective region of the radiation detection unit 15” refers to a positional relationship in which at least a part of the imaging part of the subject is out of the irradiation field 105 of radiation and at least a part of radiation transmitted through the imaging part of the subject does not reach the detection effective region.

in a case where the second recognition unit 62 recognizes the subject by template matching, the determination unit 52 can determine whether or not the subject is in the effective detection region of the radiation detection unit 15 based on information associated with a plurality of templates. In this case, determination regarding whether or not the subject is in the effective detection region of the radiation detection unit 15 can be accurately performed compared to a case where determination is performed based on information associated with one template. Information associated with a template is a classification of a part or the like of the subject represented by the template and a correlation value of the template and the camera image 121. The correlation value of each template and the camera image 121 is calculated by the second recognition unit 62. For example, in a case where the imaging part is a chest, a head is present in a predetermined direction with respect to the chest, and an abdomen is present in an opposite direction. For this reason, for example, the determination unit 52 can specify, as “chest”, a range where a correlation value with the template of the chest is equal to or greater than a first threshold value (a lower limit correlation value at which specification can be made to be the chest) and a correlation value with the template of the head is equal to or less than a second threshold value (an upper limit correlation value at which specification can be made to be not the head) and can determine whether or not the subject is in the detection effective region of the radiation detection unit 15. In this case, a boundary between the head and the chest of the subject is accurate compared to a case where the range of “chest” is specified by the correlation value with the template of the chest. The same applies to a boundary between the chest and the abdomen, or the like. As described above, the second recognition unit 62 can recognize the subject based on information associated with a plurality of templates. In this case, the determination unit 52 can accurately determine whether or not the subject is in the effective detection region of the radiation detection unit 15 merely by using a recognition result of the second recognition unit 62.

The controller 53 integrally controls the units of the radiography apparatus 10. In particular, the controller 53 performs operation support of the radiography apparatus 10 using the determination result of the determination unit 52. For example, as a result of the determination of the determination unit 52, in a case where the subject is not in the detection effective region, the controller 53 performs operation support of position adjustment for putting the subject in the detection effective region. Specifically, as shown in FIG. 8, the controller 53 comprises an imaging controller 71, a radiation controller 72, a position controller 73, and a notification unit 75.

The imaging controller 71 controls the imaging unit 35. For example, in a case where either of the first recognition unit 61 or the second recognition unit 62 requests the camera image 121, the imaging controller 71 images the subject or the like with the imaging unit 35 and provides the camera image 121 to at least one of the first recognition unit 61 or the second recognition unit 62. The imaging controller 71 moves the imaging unit 35 (in the embodiment, the distal end portion 13) in a case where the second recognition unit 62 does not recognize the subject. With this, in a case where the second recognition unit 62 does not recognize the subject, the controller 53 automatically searches the subject.

The radiation controller 72 controls the radiation source 31, the collimator 32, and the filter 36. Specifically, the radiation controller 72 comprises a radiation source controller 81, an irradiation field controller 82, and a filter insertion-extraction controller 83.

The radiation controller 72 controls the radiation source 31, and performs a start or a stop of the radiation source 31, settings of the energy, the dose, and the like of radiation generated by the radiation source 31 (in the embodiment, settings of the tube voltage and the tube current of the X-ray tube), a setting of validation or invalidation of exposure of radiation, control of an exposure timing of radiation, and the like. The control of the exposure timing is, for example, synchronization control or the like of exposure of the radiation from the radiation source 31 and the operation of the image acquisition unit 46. Validation or invalidation of exposure of radiation refers to validating or invalidating an input of an exposure instruction from the laboratory technician or the like or validating or invalidating an input of a start instruction of the radiation source 31. The radiation source controller 81 can validate or invalidate exposure of radiation using the determination result of the determination unit 52 according to the setting. With this, the controller 53 performs operation support of the radiography apparatus 10 (in particular, proper exposure control). For example, as a result of the determination of the determination unit 52, in a case where the subject is in the detection effective region, the radiation controller 72 validates exposure of radiation. This is because a state in which radiography can be executed without failure is set. On the other hand, as a result of the determination of the determination unit 52, in a case where the subject is not in the detection effective region, exposure of radiation is invalidated. This is because a state in which the subject cannot be radiographed without excess and deficiency is set, re-imaging is required, and an exposure dose of the subject increases.

The irradiation field controller 82 performs control such that the collimator 32 adjusts the irradiation field 105 of radiation. In principle, the irradiation field controller 82 makes the irradiation field 105 of radiation conform to the detection effective region of the radiation detection unit 15. The irradiation field controller 82 can change the irradiation field 105 by performing control on the collimator 32 using the determination result of the determination unit 52 according to the settings. With this, the controller 53 performs operation support of the radiography apparatus 10 (in particular, the collimator 32). For example, as a result of the determination of the determination unit 52, in a case where a part of the imaging part is out of the irradiation field 105 of an initial setting or the irradiation field 105 set by the laboratory technician or the like, the irradiation field controller 82 can put the subject (at least the imaging part) in the detection effective region by performing control such that the collimator 32 changes the irradiation field 105. This is to obtain a radiographic image of the subject without excess and deficiency by single radiography. For example, as a result of the determination of the determination unit 52, in a case where the subject is in the detection effective region, the irradiation field controller 82 performs control such that the collimator 32 changes the irradiation field in conformity with the subject according to a relationship between the subject and the size of the detection effective region. That is, in a case where the imaging part of the subject is smaller than the detection effective region, the irradiation field controller 82 narrows the irradiation field 105 in conformity with the imaging part of the subject. This is to reduce the exposure of the subject.

The filter insertion-extraction controller 83 controls insertion and extraction of the filter 36 into and from the exposure path of radiation. For example, in a case where the tube voltage set by the radiation source controller 81 is higher than a predetermined threshold value, the filter insertion-extraction controller 83 inserts the filter 36 into the exposure path of radiation. This is because, in a case where the tube voltage is set to be high, and radiation having high energy and high penetration is used for radiography, exposure to radiation having low energy and low penetration is reduced.

The position controller 73 controls the arm portion 12, the distal end portion 13, and the casters 21. Specifically, the position controller 73 comprises a radiation source position controller 91 and a main body position controller 92.

The radiation source position controller 91 controls the position of the radiation source 31 with respect to the radiation detection unit 15 by controlling folding and expanding of the arm portion 12 and the orientation of the distal end portion 13 with respect to the arm portion 12. The radiation source position controller 91 can control the position of the radiation source 31 using at least one of the recognition result of the first recognition unit 61, the recognition result of the second recognition unit 62, the determination result of the determination unit 52, or a combination thereof according to the settings. With this, the controller 53 performs operation support of the radiography apparatus 10 (in particular, the position of the radiation source 31). For example, the radiation source position controller 91 can automatically move the radiation source 31 to a position confronting the radiation detection unit 15 in a case where the first recognition unit 61 recognizes the radiation detection unit 15. With this, it is possible to reduce an operation burden of the laboratory technician or the like pertaining to alignment of the radiation source 31 and the radiation detection unit 15. The position confronting the radiation detection unit 15 refers to a position where radiation exposure can be performed to the detection effective region.

In a case of automatically moving the radiation source 31, it is preferable that the radiation source position controller 91 automatically moves the radiation source 31 in a case where the radiation detection unit 15 is substantially horizontal and stationary. This is because the disposition of the radiation detection unit 15 is completed, and thus, the radiation source 31 can be moved safely without colliding with the laboratory technician or the like. In a case of automatically moving the radiation source 31, it is preferable that the controller 53 notifies of an effect that the radiation source 31 is to be moved. This is to secure safety. The notification to the effect that the radiation source 31 is to be moved refers to a state in which the laboratory technician or the like can recognize the effect that the radiography apparatus 10 automatically moves the radiation source 31. The notification to the effect that the radiation source 31 is to be moved is performed by the notification unit 75.

The radiation source position controller 91 maintains a distance between the radiation source 31 and the radiation detection unit 15 at a specific distance. The distance between the radiation source 31 and the radiation detection unit 15 is a so-called source to image-receptor distance (SID). The specific distance is a distance appropriate for radiography according to at least one of the imaging part, the imaging conditions, or the like.

The main body position controller 92 adjusts the position and the orientation of the main body 11 in, for example, a patient's room or the like by controlling rotation of each caster 21. In a case where the radiation source position controller 91 controls the position and the like of the radiation source 31, the main body position controller 92 cooperates with the radiation source position controller 91 as necessary and adjusts the position and the orientation of the main body 11. The main body position controller 92 can make the radiography apparatus 10 automatically or semiautomatically travel in a case of carrying the radiography apparatus 10 to the patient's room or the like. Semiautomatic traveling refers to assisting the movement of the radiography apparatus 10 by rotating the casters 21 in an orientation of reducing force pushing and pulling the radiography apparatus 10.

The notification unit 75 notifies of information for supporting an operation of the radiography apparatus 10. The notification refers to a state in which the laboratory technician or the like can recognize information. The notification unit 75 notifies of the above-described information, for example, by displaying a character, a message, a figure (icon or the like), a symbol, or the like on a screen of the touch panel 23, generating sound or voice with a speaker (not shown), turning on or off an indicator (not shown), such as a lamp, or changing the display lamps. In the embodiment, the notification unit 75 performs the above-described notification by displaying a message on the screen of the touch panel 23.

The notification unit 75 can perform the above-described notification using the determination result of the determination unit 52. With this, the controller 53 performs operation support of the radiography apparatus 10. For example, the notification unit 75 notifies that the subject is in the detection effective region or that the subject is not in the detection effective region. In a case where the subject is not in the detection effective region, the notification unit 75 notifies that the subject is not in the detection effective region after the second recognition unit 62 recognizes the subject. In a case where the subject is not in the detection effective region and in a case where the subject is not recognizable by the second recognition unit 62, the notification unit 75 notifies that the subject is not recognizable.

In addition, the notification unit 75 notifies of a direction for moving the radiation detection unit 15 to put the subject in the detection effective region. The notification unit 75 notifies of a distance for moving the radiation detection unit 15 to put the subject in the detection effective region. The notification unit 75 notifies of an orientation, a direction, or an angle of rotating or inclining the radiation detection unit to put the subject in the detection effective region.

The laboratory technician or the like can performs easily adjustment (re-disposition or the like) of at least one of the subject, the radiation detection unit 15, or the radiation source 31 as necessary according to any one of various kinds of notification or a combination of a plurality of kinds of notification. As a result, the imaging part can be reliably radiographed without excess and deficiency by single imaging.

The radiography apparatus 10 configured as above operates as follows. As shown in FIG. 9, the main body 11 is moved to the patient's room that the patient 101 as a subject is present (Step S101), and the main body 11 is disposed at a position generally appropriate for radiography. The laboratory technician or the like takes out the radiation detection unit 15 from the holder 24 and disposes the radiation detection unit 15 at a position according to an imaging part (Step S102). For example, as shown in FIG. 10, the patient 101 as a subject is lying on the bed 102, and the radiation detection unit 15 is disposed between the imaging part and the bed 102. In this case, the first recognition unit 61 recognizes and traces the radiation detection unit 15 according to information obtained with the position sensor 48, and in a case where the radiation detection unit 15 is substantially horizontal and stationary, the radiation source position controller 91 automatically moves the radiation source 31 at a position confronting the radiation detection unit 15 (Step S103). When necessary, the main body position controller 92 automatically adjusts the position of the main body 11. As a result, a distance D1 between the generation point 103 of radiation and the radiation detection unit 15 becomes the SID set in advance according to the imaging part and the like, and the irradiation field 105 of radiation becomes a range including the detection effective region of the radiation detection unit 15. A distance D2 is a so-called source to object distance (SOD), and a distance D3 is a thickness (body thickness) of a body in the imaging part of the patient 101.

In a case where the radiation detection unit 15 and the radiation source 31 are disposed as described above, the first recognition unit 61 recognizes the radiation detection unit 15 (Step S104), and the second recognition unit 62 recognizes the patient 101 as a subject (Step S105).

That is, the imaging unit 35 images the patient 101 and the like and provides the camera image 121 to the first recognition unit 61 and the second recognition unit 62. Then, in a case where the radiation detection unit 15 is captured in the camera image 121, the first recognition unit 61 specifies the position and the like of the radiation detection unit 15 using at least one of the camera image 121 or information obtained with the position sensor 48. In a case where the radiation detection unit 15 is not captured in the camera image 121, such as a case where the radiation detection unit 15 is behind the patient 101, or in a case where the radiation detection unit 15 is not captured to a degree enough for use in recognition of the radiation detection unit 15, the first recognition unit 61 specifies the position and the like of the radiation detection unit 15 using information obtained with the position sensor 48. With this, as a result, the first recognition unit 61 recognizes the detection effective region. The second recognition unit 62 specifies the position and the like of the patient 101, in particular, the imaging part of the patient 101 using the camera image 121.

In a case where the first recognition unit 61 and the second recognition unit 62 end the recognition processing, the determination unit 52 determines whether or not the imaging part of the patient 101 is in the detection effective region using the recognition results of the first recognition unit 61 and the second recognition unit 62 (Step S106).

In a case where the imaging part of the patient 101 is in the detection effective region (Step S106: YES), the irradiation field controller 82 determines whether or not the irradiation field 105 is appropriate using the determination result (Step S107). In a case where the irradiation field 105 is appropriate (Step S107: YES), the radiation source controller 81 validates exposure of radiation (Step S108). In a case where the irradiation field 105 is not appropriate (Step S107: NO), the irradiation field controller 82 automatically adjusts the irradiation field 105 (Step S108), and thereafter, the radiation source controller 81 validates exposure of radiation. For example, in a case where the imaging part is the chest 122 of the patient 101, and as shown in FIG. 10, in a camera image 121, in a case where the chest 122 is small with respect to the detection effective region (a region indicated by a marker 42), and a blank is large in a radiographic image, the irradiation field controller 82 adjusts the irradiation field 105 to a region 123 conforming to the chest 122. In a case where exposure of radiation is validated, the radiation source controller 81 automatically sets imaging conditions and waits for an input of an exposure instruction from the laboratory technician or the like. Then, in a case where there is an input of the exposure instruction from the laboratory technician or the like, the radiation source controller 81 performs radiation exposure from the radiation source 31, the radiation detection unit 15 acquires a radiographic image (Step S111).

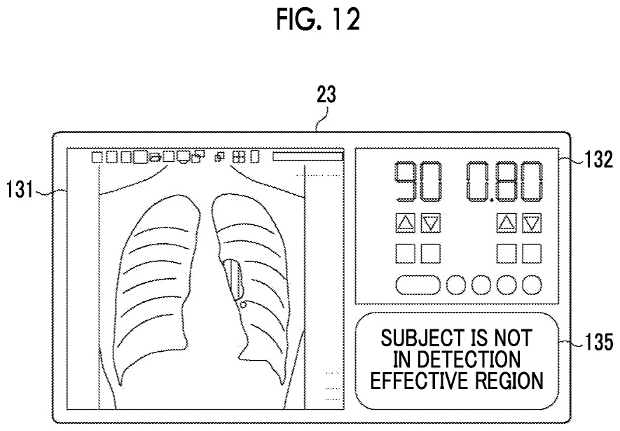

On the other hand, in a case where the imaging part of the patient 101 is not in the detection effective region (Step S106: NO), the notification unit 75 notifies of the effect by displaying a message on the screen of the touch panel 23 (Step S112). For example, as shown in FIG. 12, a console 131 where the radiographic image and the like are displayed and an imaging condition setting portion 132 where the imaging conditions, such as the tube voltage and the tube current, are set are displayed on the screen of the touch panel 23, and the notification unit 75 further displays a message 135 to the effect that the imaging part of the patient 101 is not in the detection effective region. When viewing the message 135, the laboratory technician or the like can know that the imaging part of the patient 101 is not in the detection effective region, and radiography may fail. Accordingly, the laboratory technician or the like re-disposes the radiation detection unit 15 with respect to the patient 101 by moving the radiation detection unit 15 or the patient 101 (Step S114). In a case where the imaging part of the patient 101 is not in the detection effective region, the radiation source controller 81 invalidates exposure of radiation (Step S113), and prevents erroneous exposure of radiation. In a case where re-disposition of the radiation detection unit 15 and the like is performed, the first recognition unit 61 and the second recognition unit 62 execute the recognition processing again. This loop is repeated until the determination unit 52 determines that the imaging part of the patient 101 is in the detection effective region.

As described above, the radiography apparatus 10 recognizes the radiation detection unit 15 and the patient 101 (in particular, the imaging part) as a subject and determines whether or not the patient 101 is in the detection effective region. Then, the controller 53 performs the adjustment of the irradiation field 105, the validation or invalidation of exposure, the notification of the message for prompting re-disposition, and the like using the determination result to perform operation support of the radiography apparatus 10. As a result, with the radiography apparatus 10, it is possible to support reliable radiography even though the radiation source 31 and the radiation detection unit 15 are movable independently. That is, it is possible to reliably obtain a radiographic image of an imaging part to be imaged by single radiography.

The movement of the radiation source 31 of Step S103 can be performed manually. Even though the radiation source 31 is manually moved to the position confronting the radiation detection unit 15, the radiation source position controller 91 may automatically make fine adjustment on the position of the radiation source 31. This is because accurate alignment (including the adjustment of the SID) can be supported. Similarly, the position adjustment of the main body 11 can be performed manually. Even though the main body 11 is manually moved, the main body position controller 92 may automatically make fine adjustment on the position of the main body 11. This is because accurate alignment can be supported. In addition, at least one of the setting of the imaging conditions by the radiation source controller 81, insertion and extraction of the filter 36 by the filter insertion-extraction controller 83, and the like can be performed manually, and even though these are manually set or the like, it is possible to automatically perform fine adjustment of setting and the like. This is because accurate setting and the like are supported. That is, a part of the matters that are performed automatically by the radiography apparatus 10 in the above-described first embodiment can be performed manually. Furthermore, it is possible to support accurate setting and the like of setting matters and the like, which are performed manually, through fine adjustment or the like in the radiography apparatus 10.

### Second Embodiment

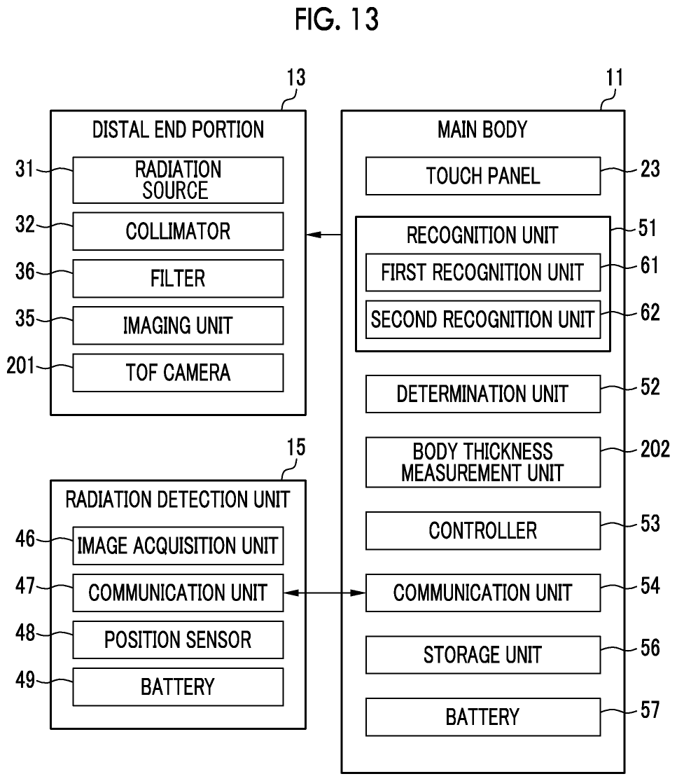

In the first embodiment, although the imaging unit 35 is provided in the distal end portion 13, as shown in FIG. 13, a time of flight camera (TOF camera) 201 can be provided instead of the imaging unit 35 or in addition to the imaging unit 35.

The TOF camera 201 is a camera that emits near infrared light in a pulsed manner and measures a reflection time of near infrared light from the subject. For this reason, in a case where the TOF camera 201 is provided, a body thickness measurement unit 202 that measures a body thickness (distance D3) of the imaging part of the patient 101 using an image (hereinafter, referred to as a distance image) output from the TOF camera 201 is provided in, for example, the main body 11. The distance image is, for example, an image in which each pixel has a pixel value having correlation with a distance from the TOF camera 201. The body thickness measurement unit 202 measures the body thickness (distance D3) distance image, by obtaining the distance D2 from the generation point 103 of radiation to the imaging part of the patient 101 and subtracting the distance D2 from the distance D1 as the SID.

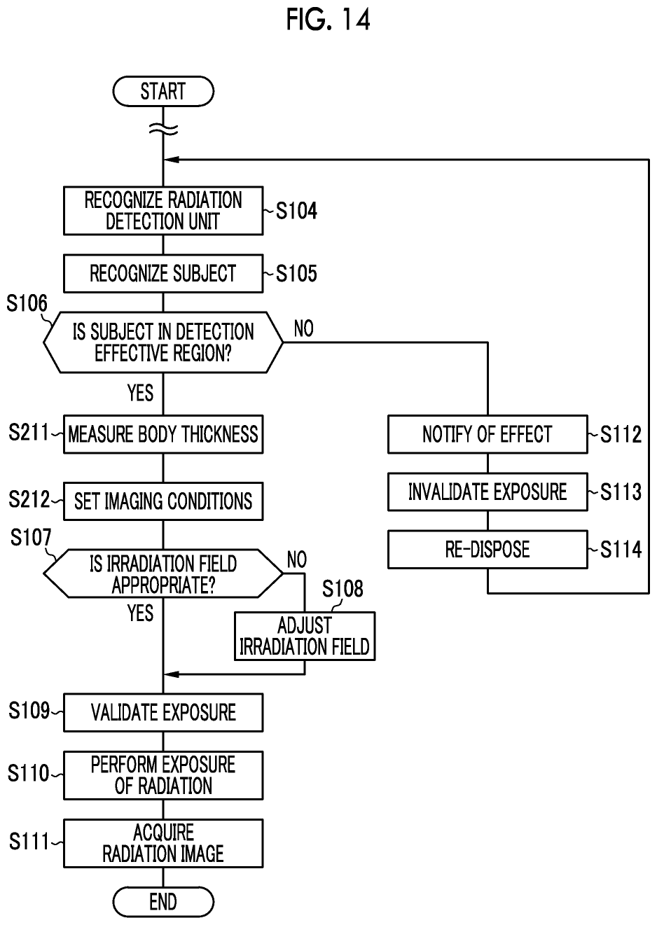

In a case where the radiography apparatus 10 has the TOF camera 201, and the body thickness measurement unit 202 measures the body thickness of the subject, as shown in FIG. 14, for example, after the determination (Step S106) regarding whether or not the subject is in the detection effective region, a body thickness measurement step S211 of measuring the body thickness of the subject and an imaging condition setting step S212 of setting imaging conditions using the body thickness of the subject can be included. The body thickness measurement step S211 is a step including capturing of the distance image by the TOF camera 201 and body thickness measurement by the body thickness measurement unit 202. The imaging condition setting step S212 is a step in which, in a case where the subject is in the detection effective region, the controller 53 automatically sets the imaging conditions related to the body thickness of the subject using the body thickness of the subject. The setting of the imaging conditions includes changing or adjusting an imaging condition (a preset imaging condition or a manually set imaging condition) already set, in addition to newly setting an imaging condition. The imaging conditions related to the body thickness of the subject are, for example, the setting of the tube voltage of the radiation source 31 (in the embodiment, the X-ray tube), insertion and extraction of the filter 36, and the like. That is, in the imaging condition setting step S212, the controller 53 sets the tube voltage of the radiation source 31 using the body thickness of the subject. Furthermore, in the imaging condition setting step S212, the controller 53 inserts or extracts the filter 36 between the radiation source 31 and the subject using the body thickness of the subject.

More specifically, the radiation source controller 81 increases the tube voltage according to the body thickness of the subject in the imaging condition setting step S212. With this, penetration of radiation is increased, and even though the body thickness of the subject is large, a clear radiographic image is obtained. On the other hand, in the imaging condition setting step S212, the filter insertion-extraction controller 83 inserts the filter 36 into the exposure path in a case where the tube voltage is equal to or greater than a predetermined threshold value. With this, exposure to the subject is reduced by shielding radiation having low penetration and low energy that does not contribute to a radiographic image.

As described above, in a case where the radiography apparatus 10 is configured to measure the body thickness of the patient 101 as the subject, the controller 53 can perform operation support of the radiography apparatus 10 using the determination result of the determination unit 52 and the body thickness of the patient 101 measured by the body thickness measurement unit 202. Specifically, the controller 53 can automatically and appropriately set the imaging conditions using the body thickness of the patient 101 as the subject as described above in a case where the patient 101 as the subject is in the detection effective region.

### Third Embodiment

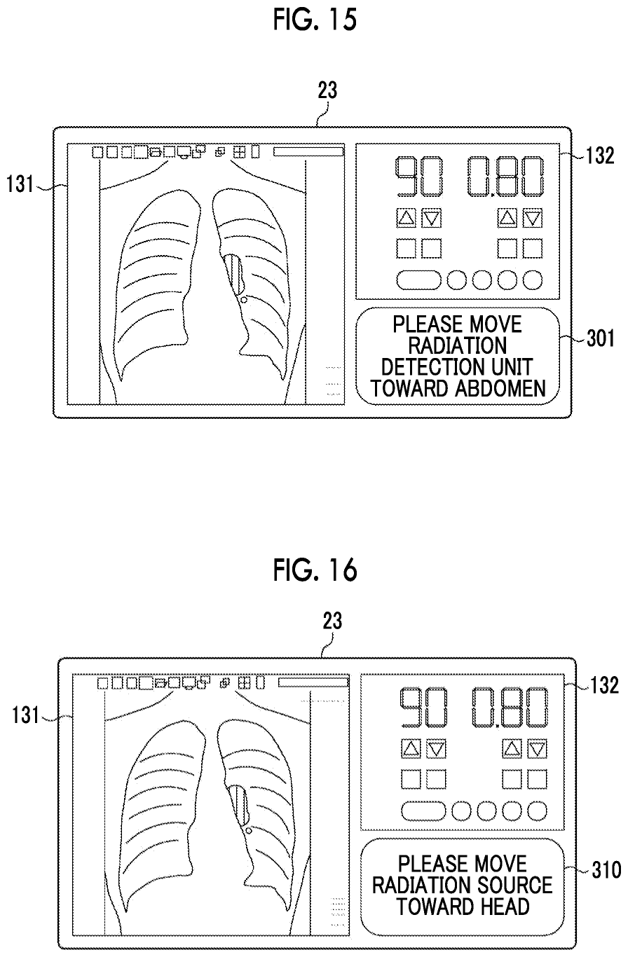

In the above-described first embodiment and the like, in a case where the subject is not in the effective detection region, the effect is notified to prompt re-disposition (Step S114). In contrast, in a case where re-disposition is required, the radiography apparatus 10 can more efficiently support re-disposition of the radiation source 31, the radiation detection unit 15, or the subject. For example, as shown in FIG. 15, the controller 53 can notify of a direction for moving the radiation detection unit 15 with respect to the subject using a message 301 or the like with the notification unit 75. When viewing the message 301, the laboratory technician or the like can easily re-dispose the radiation detection unit 15 with respect to the subject compared to a case where the message 301 is not provided. As a result, it is possible to put the subject in the effective detection region in a short time and accurately.

In addition, the controller 53 can notify of a distance for moving the radiation detection unit 15 with the notification unit 75 as well as the moving direction of the radiation detection unit 15. In this case, it is possible to put the subject in the effective detection region in a shorter time and more accurately compared to a case where only the direction for moving the radiation detection unit 15 is notified.

As described above, in a case where re-disposition of the radiation detection unit 15 and the like is required, the controller 53 notifies of the direction and the distance for moving the radiation detection unit 15 with the notification unit 75. This is particularly useful in a case where the controller 53 automatically moves the radiation source 31 to the position confronting the radiation detection unit 15 with the radiation source position controller 91. This is because the radiation source position controller 91 automatically re-disposes the radiation source 31 following re-disposition of the radiation detection unit 15, and thus, with just moving the radiation detection unit 15, it is possible to put the subject in the effective detection region and re-disposition is completed.

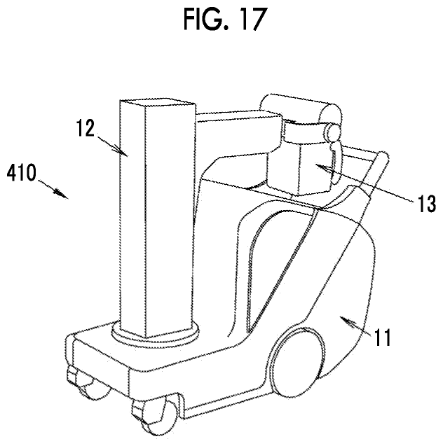

In a case of manually moving the radiation source 31, as shown in FIG. 16, the controller 53 can notify of a direction for moving the radiation source 31 using a message 310 or the like with the notification unit 75. In addition, the controller 53 can further notify of a distance for moving the radiation source 31 with the notification unit 75. With this, it is possible to easily complete re-disposition even in a case of manually moving the radiation source 31.

In a case of manually moving the radiation source 31, the controller 53 can notify that the radiation source 31 is at the position confronting the radiation detection unit 15, using message display, voice, light, or other methods with the notification unit 75. In a case where the notification unit 75 notifies that the radiation source 31 is at the position confronting the radiation detection unit 15, the laboratory technician or the like can more easily complete re-disposition of the radiation source 31.

In a case of manually moving the radiation source 31, the controller 53 can regulate a moving direction of the radiation source 31 with the radiation source position controller 91. For example, the radiation source position controller 91 permits movement in a direction in which the radiation source 31 approaches the position confronting the radiation detection unit 15, but regulates movement in a direction in which the radiation source 31 goes away from the position confronting the radiation detection unit 15. With this, the laboratory technician or the like can complete re-disposition of the radiation source 31 by just moving the radiation source 31 in a direction in which the radiation source 31 can be naturally moved. The radiation source position controller 91 can physically regulate the movement of the radiation source 31 by an electromagnetic lock or the like of the arm portion 12. In addition, the radiation source position controller 91 can regulate substantial movement of the radiation source 31 by notification of an alert with the notification unit 75, guidance of the moving direction of the radiation source 31, or the like.

In the above-described third embodiment, although an operation support aspect on the re-disposition of the radiation detection unit 15 and the radiation source 31 has been described, in a case where the subject can be moved, the radiography apparatus 10 can perform support of notification of a moving direction or the like as above for the subject.

In the above-described third embodiment, although a case where at least one of the radiation detection unit 15, the radiation source 31, or the subject is re-disposed has been described, operation support of the third embodiment can also be performed in a case of initially disposing the radiation detection unit 15 or the like. That is, the invention is not limited to a case of re-disposing the radiation detection unit 15 or the like, and in a case of manually moving the radiation source 31, the controller 53 can notify that the radiation source 31 is at the position confronting the radiation detection unit 15. In addition, in a case of manually moving the radiation source 31, the controller 53 can notify of a direction for moving the radiation source 31. The same applies to others. In any cases, disposition of at least one of the radiation detection unit 15, the radiation source 31, or the subject is facilitated.

In the first embodiment, the second embodiment, and the third embodiment, and the like described above, in a case where the radiation detection unit 15 is attached to the main body 11 including the controller 53 (a case where the radiation detection unit 15 is in the holder 24), the controller 53 restricts the movement of the radiation source 31. That is, the radiation source position controller 91 does not move the radiation source 31 in a case where the radiation detection unit 15 is attached to the main body 11 including the controller 53. In addition, in a case where the radiation source 31 is movable manually, the radiation source position controller 91 restricts the movement of the radiation source 31. This is to secure safety, for example, to prevent the distal end portion 13 including at least one of the arm portion 12 or the radiation source 31 from colliding with the laboratory technician, the subject, other hospital equipment, or the like.

In a case where the main body 11 including the controller 53 is moving, the controller 53 restricts the movement of the radiation source 31. That is, the radiation source position controller 91 does not move the radiation source 31 in a case where the main body 11 is moving. In addition, in a case where the radiation source 31 is movable manually, the radiation source position controller 91 restricts the movement of the radiation source 31. This is to secure safety, for example, to prevent the distal end portion 13 including at least one of the arm portion 12 or the radiation source 31 from colliding with the laboratory technician, the subject, other hospital equipment, or the like as above.

In a case where a sensor that detects a surrounding object, such as an infrared sensor, is provided in the distal end portion 13 or the like, the controller 53 may restrict the movement of the radiation source 31 in a case where the sensor detects a surrounding object. The reason is the same as above.

In the first embodiment, the second embodiment, the third embodiment, and the like described above, although the imaging unit 35 is provided in the distal end portion 13, the imaging unit 35 can be provided in any portion other than the distal end portion 13 as long as the subject in the irradiation field 105 can be imaged.

In the first embodiment, the second embodiment, the third embodiment, and the like described above, although the arm portion 12 is a foldable type, the invention can be applied to a radiography apparatus 401 in which, as shown in FIG. 17, an arm portion 12 moves rotationally and expands and contracts (so-called telescopic arm). Although the radiography apparatus 10 and the radiography apparatus 410 are so-called treatment carts, the invention can be applied to a radiography apparatus or the like in which a part of the configuration of the radiography apparatus 10 and the like is fixed in a laboratory or the like.

The above-described first embodiment and the like include a radiography system comprising the radiation source 31 that generates radiation, the radiation detection unit 15 that is movable independently with respect to the radiation source 31 and obtains the image of the subject by detecting radiation transmitted through the subject, the imaging unit 35 that images at least the subject using light having a wavelength longer than radiation, the first recognition unit 61 that recognizes the radiation detection unit 15, the second recognition unit 62 that recognizes the subject using the image captured by the imaging unit 35, the determination unit 52 that, using the recognition results of the first recognition unit 61 and the second recognition unit 62, specifies the relative positional relationship between the radiation detection unit 15 and the subject and determines whether or not the subject is in the detection effective region of the radiation detection unit 15, and the controller 53 that performs operation support using the determination result of the determination unit 52. The radiography system is not limited to a mobile type (treatment cart), and includes a system that has a part of components fixed in a laboratory or the like.

The above-described first embodiment and the like include an operation method of the radiography apparatus or the radiography system having the radiation source 31 that generates radiation, the radiation detection unit 15 that is movable independently with respect to the radiation source 31 and obtains the image of the subject by detecting radiation transmitted through the subject, and the imaging unit 35 that images at least the subject using light having a wavelength longer than radiation, the operation method of the radiography apparatus or the radiography system comprising a step in which the first recognition unit 61 recognizes the radiation detection unit 15, a step in which the second recognition unit 62 recognizes the subject using the image captured by the imaging unit 35, a step in which, using the recognition results of the first recognition unit 61 and the second recognition unit 62, the determination unit 52 specifies the relative positional relationship between the radiation detection unit 15 and the subject and determines whether or not the subject is in the detection effective region of the radiation detection unit 15, and a step in which the controller 53 performs operation support using the determination result of the determination unit 52.

In the above-described embodiments and the like, the hardware structures of the processing units that execute various kinds of processing, such as the recognition unit 51 (the first recognition unit 61 and the second recognition unit 62) and the controller 53 (the imaging controller 71, the radiation controller 72, the position controller 73, the notification unit 75, and each unit constituting them), are various processors described below. Various processors include a central processing unit (CPU) that is a general-purpose processor executing software (program) to function as various processing units, a programmable logic device (PLD) that is a processor capable of changing a circuit configuration after manufacture, such as a graphical processing unit (GPU) or a field programmable gate array (FPGA), a dedicated electric circuit that is a processor having a circuit configuration dedicatedly designed for executing various kinds of processing, and the like.

One processing unit may be configured of one of various processors described above or may be configured of a combination of two or more processors (for example, a plurality of FPGAs, a combination of a CPU and an FPGA, a combination of a CPU and a GPU, or the like) of the same type or different types. A plurality of processing units may be configured of one processor. As an example where a plurality of processing units are configured of one processor, first, as represented by a computer, such as a client or a server, there is a form in which one processor is configured of a combination of one or more CPUs and software, and the processor functions as a plurality of processing units. Secondly, as represented by system on chip (SoC) or the like, there is a form in which a processor that implements all functions of a system including a plurality of processing units into one integrated circuit (IC) chip is used. In this way, various processing units may be configured using one or more processors among various processors described above as a hardware structure.

In addition, as the hardware structure of various processors, more specifically, an electric circuit (circuitry), in which circuit elements, such as semiconductor elements, are combined can be used.

### EXPLANATION OF REFERENCES

10, 401: radiography apparatus
11: main body
12: arm portion
13: distal end portion
15: radiation detection unit
21: caster
22: grip portion
23: touch panel
24: holder
31: radiation source
32: collimator
33: handle
35: imaging unit
36: filter
41, 42: marker
43: position sensor
46: image acquisition unit
47, 54: communication unit
48: position sensor
49,57: battery
51: recognition unit
52: determination unit
53: controller
56: storage unit
61: first recognition unit
62: second recognition unit
64: template
64a: chest
65A, 65B: comparison range
71: imaging controller
72: radiation controller
73: position controller
75: notification unit
81: radiation source controller
82: irradiation field controller
83: filter insertion-extraction controller
91: radiation source position controller
92: main body position controller
101: patient
102: bed
103: generation point
105: irradiation field
121: camera image
122: chest
123: region
131: console
132: imaging condition setting portion
135, 301, 310: message
201: TOF camera
202: body thickness measurement unit
D1, D2, D3: distance
S101 to S111, S211, S212: step of operation

What is claimed is:

1. A radiography apparatus comprising:
a radiation source that generates radiation;
a radiation detection device that is movable independently with respect to the radiation source and obtains an image of a subject by detecting the radiation transmitted through the subject;
an imaging device that images at least the subject using light having a wavelength longer than the radiation;
a processor configured to function as:
a first recognition unit that recognizes the radiation detection device;
a second recognition unit that recognizes the subject using an image captured by the imaging device;
a determination unit that, using recognition results of the first recognition unit and the second recognition unit, specifies a relative positional relationship between the radiation detection device and the subject and determines whether or not the subject is in a detection effective region of the radiation detection device; and
a controller that performs operation support using a determination result of the determination unit.

2. The radiography apparatus according to claim 1,
wherein the controller notifies that the subject is in the detection effective region or that the subject is not in the detection effective region.

3. The radiography apparatus according to claim 2,
wherein, in a case where the subject is not in the detection effective region, the controller notifies that the subject is not in the detection effective region after the second recognition unit recognizes the subject.

4. The radiography apparatus according to claim 2,
wherein, in a case where the subject is not in the detection effective region and the subject is not recognizable by the second recognition unit, the controller notifies that the subject is not recognizable.

5. The radiography apparatus according to claim 1,
wherein the controller validates or invalidates exposure of the radiation using the determination result.

6. The radiography apparatus according to claim 5,
wherein the controller validates the exposure of the radiation in a case where the subject is in the detection effective region.

7. The radiography apparatus according to claim 5,
wherein the controller invalidates the exposure of the radiation in a case where the subject is not in the detection effective region.

8. The radiography apparatus according to claim 1,
wherein the controller performs operation support of position adjustment for putting the subject in the detection effective region in a case where the subject is not in the detection effective region.

9. The radiography apparatus according to claim 8,
wherein the controller notifies of a direction for moving the radiation detection device to put the subject in the detection effective region.

10. The radiography apparatus according to claim 8,
wherein the controller notifies of an orientation, a direction, or an angle for rotating or inclining the radiation detection device to put the subject in the detection effective region.

11. The radiography apparatus according to claim 1, further comprising:
a collimator that defines an irradiation field of the radiation,
wherein the controller performs control using the determination result that the collimator adjusts the irradiation field.

12. The radiography apparatus according to claim 1, further comprising:
a collimator that defines an irradiation field of the radiation,
wherein the controller puts the subject in the detection effective region by performing control using the determination result such that the collimator changes the irradiation field.

13. The radiography apparatus according to claim 1, further comprising:
a collimator that defines an irradiation field of the radiation,
wherein the controller performs control such that the collimator changes the irradiation field in conformity with the subject in a case where the subject is in the detection effective region.

14. The radiography apparatus according to claim 1,
wherein the first recognition unit specifies a position and an orientation of the radiation detection device.

15. The radiography apparatus according to claim 14,
wherein the first recognition unit recognizes the radiation detection device using the image acquired by the imaging device.

16. The radiography apparatus according to claim 15,
wherein the radiation detection device has a marker indicating a position, and
the first recognition unit recognizes a position and an orientation of the radiation detection device using the marker in the image acquired by the imaging device.

17. The radiography apparatus according to claim 14,
wherein the radiation detection device comprises a position sensor that measures a position of the radiation detection device, and
the first recognition unit recognizes the radiation detection device using information obtained by the position sensor.

18. The radiography apparatus according to claim 1,
wherein the second recognition unit recognizes a part of the subject.

19. The radiography apparatus according to claim 1,
wherein the controller automatically moves the radiation source to a position confronting the radiation detection device in a case where the first recognition unit recognizes the radiation detection device.

20. The radiography apparatus according to claim 19,
wherein the controller automatically moves the radiation source in a case where the radiation detection device is horizontal and stationary.

21. The radiography apparatus according to claim 19,
wherein, in a case of automatically moving the radiation source, the controller notifies of an effect that the radiation source is to be moved.

22. The radiography apparatus according to claim 19,
wherein the controller maintains a distance between the radiation source and the radiation detection device at a specific distance.

23. The radiography apparatus according to claim 1,
wherein the controller moves the imaging device in a case where the second recognition unit does not recognize the subject.

24. The radiography apparatus according to claim 1, further comprising:
a body thickness measurement device that measures a body thickness of the subject,
wherein the controller performs operation support using the determination result and the body thickness of the subject.

25. The radiography apparatus according to claim 24,
wherein the controller sets an imaging condition using the body thickness of the subject in a case where the subject is in the detection effective region.

26. The radiography apparatus according to claim 25,
wherein the controller sets a tube voltage of the radiation source using the body thickness of the subject.

27. The radiography apparatus according to claim 25,
wherein the controller inserts or extracts a filter shielding a part of the radiation between the radiation source and the subject using the body thickness of the subject.

28. The radiography apparatus according to claim 1,
wherein, in a case of manually moving the radiation source, the controller notifies that the radiation source is at a position confronting the radiation detection device.

29. The radiography apparatus according to claim 1,
wherein, in a case of manually moving the radiation source, the controller notifies of a direction for moving the radiation source.

30. The radiography apparatus according to claim 1,
wherein, in a case where the radiation detection device is attached to a main body including the controller, the controller restricts movement of the radiation source.

31. The radiography apparatus according to claim 1,
wherein, in a case where the radiography apparatus is a mobile type and a main body including the controller is moving, the controller restricts movement of the radiation source.
